(19) United States
(12) Patent Application Publication
(10) Pub. No.: US 2012/0021401 A1
(43) Pub. Date: Jan. 26, 2012

(54) DISPOSABLE CARTRIDGE FOR AUTOMATIC AND CONTINUOUS WATER QUALITY MONITORING

(75) Inventors: Nirit Ulitzur, Kiryat-Tivon (IL); David Natan Arlinsky, Yoqne'am Illit (IL)

(73) Assignee: CHECK LIGHT LTD., Kiryat-Tivon (IL)

(21) Appl. No.: 13/146,281

(22) PCT Filed: Jan. 31, 2010
(86) PCT No.: PCT/IL10/00077

§ 371 Date: Jul. 26, 2011

Related U.S. Application Data
(60) Provisional application No. 61/148,072, filed on Jan. 29, 2009.

(51) Int. Cl. C12Q 3/00; C12Q 1/02; C12M 1/38; C12M 1/34; C12M 1/36

(52) U.S. Cl. 435/3; 435/288.7; 435/286.5; 435/287.3; 435/29

(57) ABSTRACT

A disposable cartridge for use in assaying an analyte in an automated water quality monitoring analyzer comprising: (a) a platform; (b) array of containers for separately containing at least one reagent, a buffer and a so freeze dried preparation of luminescent bacteria, said containers being mounted on said platform; (c) reaction chamber mounted on said platform and configured for being moved between said containers, such that said reagent, said buffer and said freeze dried preparation of luminescent bacteria are conveyed to said reaction chamber in a predetermined sequence such that light detectable by said analyzer is emitted; (d) fluid transferring means for fluid transfer and mixing between said containers, reaction chamber, and analyzer; (e) conveying means for moving said cartridge to predetermined positions within said automated water quality monitoring analyzer for enabling analysis of said analyte.

### BACKGROUND

Most analyzers consist of pumps, valves, tubing, chemical reagents, sensors, data collection and data communication systems, and provide frequent precise measurements that are of laboratory quality.

A long felt unmet need exists for a disposable sealed cartridge for use with automated water quality monitoring analyzers which are based on bioluminescence technology.

U.S. Pat. No. 5,801,052 describes an apparatus for reconstituting dried bacteria, but does not offer solutions to the biofouling problem mentioned above.

US 2002/0054828A1 describes an analysis device preferably in the form of a floating buoy which has a main body portion and a removable cartridge containing consumable ingredients for use in analysis, however no provision is made for use with sensitive biological reagents and bioluminescent bacteria.

### SUMMARY

It is an object of the present invention to disclose a disposable cartridge for use in assaying an analyte in an automated water quality monitoring analyzer comprising a platform; an array of containers each separately containing reagents, buffers and freeze dried preparations of luminescent bacteria, the containers mounted on the platform; a fluid transferring means for fluid transfer and mixing between the containers and the analyzer; and a plurality of connecting means adapted for connecting between the containers and the analyzer.

It is a core purpose of the invention to provide the cartridge adapted for conveyance to predetermined positions in the analyzer. The cartridge is further adapted for sequential manipulation of the containers and their contents such that the analyte can be assayed in the analyzer.

Another object of the present invention is to disclose a cartridge as defined in the above which comprises at least one container containing assay buffer, at least one container containing freeze dried luminescent bacteria and at least one assay chamber, the assay chamber adapted for reacting the analyte, the reagents, the freeze dried suspensions of luminescent bacteria and the assay buffer together such that light detectable by the analyzer is emitted.

A further object of the present invention is to disclose a cartridge as defined in any of the above in which the array additionally comprises at least one container containing disinfection buffer (DBC) mounted on the platform.

A further object of the present invention is to disclose a cartridge as defined in any of the above in which the array additionally comprises at least one container containing reference water (RWC) mounted on the platform.

A further object of the present invention is to disclose a cartridge as defined in any of the above in which the array additionally comprises a chamber for bacterial suspension.

A further object of the present invention is to disclose a cartridge as defined in any of the above in which any of the containers are provided with volume sensors, the sensors provided with logical coupling means to a microprocessor in the analyzer.

A further object of the present invention is to disclose a cartridge as defined in any of the above in which the sensors are adapted to signal data concerning contents of the containers and chambers to the microprocessor in the analyzer.

A further object of the present invention is to disclose a cartridge as defined in any of the above in which the platform is adapted for traveling on a gantry, rail, lift, carriage or track.

A further object of the invention is to disclose the use of a conveyer belt or conveying system to bring the reaction chamber to the various reagents. This is a dramatic change from the common design in which multiple tubes linked to the various reagents containers feed the reaction chamber. This novel design reduces the number of tubes which are prone to clogging due to biofouling.

A further object of the present invention is to disclose a cartridge as defined in any of the above in which at least one of the containers, chambers and fluid transferring means is adapted for manipulation by mechanical means.

A further object of the present invention is to disclose a cartridge as defined in any of the above in which the containers, chambers, syringes and fluid transferring means are attached by locking and mounting means to the platform at specific locations on the platform.

A further object of the present invention is to disclose a cartridge as defined in any of the above in which the platform is adapted to lock into the analyzer in a predetermined configuration

A further object of the present invention is to disclose a cartridge as defined in any of the above in which the platform is adapted to be temperature controlled at specific container and chamber mounting locations, thereby holding the containers and chambers at predetermined temperatures.

A further object of the present invention is to disclose a cartridge as defined in any of the above in which the platform is adapted to be cooled at specific container locations, thereby cooling the containers and chambers to predetermined temperatures.

A further object of the present invention is to disclose a cartridge as defined in any of the above in which the fluid transferring means are selected from a group consisting of inlet ports, outlet ports, syringes, valves, taps and connectors.

A further object of the present invention is to disclose a cartridge as defined in any of the above in which the array of containers additionally comprises at least one container filled with storage buffer.

A further object of the present invention is to disclose a cartridge as defined in any of the above in which the array of containers additionally comprises at least one container at least one container for “organic” buffer, the organic buffer optimized for use in assays of organic analytes.

A further object of the present invention is to disclose a cartridge as defined in any of the above in which the array of containers additionally comprises at least one container for metallic buffer, the “metallic” buffer optimized for use in assays of cationic heavy metals and metalloid analytes.

A further object of the present invention is to disclose a cartridge as defined in any of the above in which the cartridge is adapted for storage in a module of the analyzer for predetermined periods of about one month.

A further object of the present invention is to disclose a cartridge as defined in any of the above in which the cartridge is adapted for storage of in the module of the analyzer for a period of about 1-3 months.

A further object of the present invention is to disclose a cartridge as defined in any of the above in which the cartridge is presented in kit form for assembly by the user according to instructions associated with the kit.

A further object of the present invention is to disclose a cartridge as defined in any of the above in which the array of containers are provided with reagents and buffers adapted for use in assays selected from a group consisting of fluorometric assays, chemiluminescent assays and colourimetric assays.

It is yet another object of the present invention is to disclose a method of assaying an analyte in a water quality analyzer in which the method comprises steps of obtaining a disposable cartridge comprising a platform; an array of containers each separately containing reagents, buffers and freeze dried preparations of luminescent bacteria; a fluid transferring means adapted for fluid transfer and mixing between the containers and the analyzer; connecting means adapted for connecting between the containers and the analyzer; in which the assay chamber is adapted for conveying to predetermined positions in the analyzer, the cartridge further adapted for sequential manipulation of at least some of the containers and their contents such that an analyte can be assayed in the analyzer; installing the cartridge into the automated water quality monitoring analyzer; operating the automatic analyzer thereby conveying the assay chamber within the cartridge to the predetermined locations in the analyzer and sequentially manipulating the contents of the containers so as to activate the luminescent bacteria such that light is emitted; detecting the emitted light; and processing the results obtained thereby assaying the analyte.

A further object of the present invention is to disclose a method of assaying an analyte in a water quality analyzer as defined in any of the above, which comprises further steps of obtaining and installing the disposable cartridge having the array additionally comprising at least one container containing disinfection buffer (DBC) mounted on the platform.

A further object of the present invention is to disclose a method of assaying an analyte in a water quality analyzer, wherein the disposable cartridge has an array that includes at least one container containing disinfection buffer mounted on the platform; A further object of the present invention is to disclose a method of assaying an analyte in a water quality analyzer as defined in any of the above, which comprises further steps of obtaining and installing the cartridge having the array additionally comprising at least one container containing reference water (RWC) mounted on the platform.

A further object of the present invention is to disclose a method of assaying an analyte in a water quality analyzer as defined in any of the above, which comprises further steps of obtaining and installing the cartridge having the array additionally comprising a chamber for bacterial suspension.

A further object of the present invention is to disclose a method of assaying an analyte in a water quality analyzer as defined in any of the above, which comprises further steps of obtaining and installing the cartridge having any of the containers provided with volume sensors, the sensors provided with logical coupling means to a microprocessor in the analyzer.

A further object of the present invention is to disclose a method of assaying an analyte in a water quality analyzer as defined in any of the above, which comprises further steps of obtaining and installing the cartridge having the sensors adapted to signal data concerning contents of the containers and chambers to the microprocessor in the analyzer.

A further object of the present invention is to disclose a method of assaying an analyte in a water quality analyzer as defined in any of the above, which comprises further steps of obtaining and installing the cartridge having the platform adapted for traveling on a gantry, rail, lift, carriage or track.

A further object of the present invention is to disclose a method of assaying an analyte in a water quality analyzer as defined in any of the above, which comprises further steps of obtaining and installing the cartridge having at least one of the containers, chambers and fluid transferring means is adapted for manipulation by mechanical means.

A further object of the present invention is to disclose a method of assaying an analyte in a water quality analyzer as defined in any of the above, which comprises further steps of obtaining and installing the cartridge having the containers, chambers, syringes and fluid transferring means attached by locking and mounting means to the platform at specific locations on the platform.

A further object of the present invention is to disclose a method of assaying an analyte in a water quality analyzer as defined in any of the above, which comprises further steps of obtaining and installing the cartridge having the platform adapted to lock into the analyzer in a predetermined configuration.

A further object of the present invention is to disclose a method of assaying an analyte in a water quality analyzer as defined in any of the above, which comprises further steps of obtaining and installing the cartridge having the platform adapted to be temperature controlled at specific container and chamber mounting locations, thereby holding the containers and chambers at predetermined temperatures.

A further object of the present invention is to disclose a method of assaying an analyte in a water quality analyzer as defined in any of the above, which comprises further steps of obtaining and installing the cartridge having the platform adapted to be cooled at specific container locations, and cooling the containers and chambers to predetermined temperatures.

A further object of the present invention is to disclose a method of assaying an analyte in a water quality analyzer as defined in any of the above, which comprises further steps of obtaining and installing the cartridge having fluid transferring means selected from a group consisting of inlet ports, outlet ports, syringes, valves, taps and connectors and transferring fluid via the fluid transferring means.

A further object of the present invention is to disclose a method of assaying an analyte in a water quality analyzer as defined in any of the above, which comprises further steps of obtaining and installing the cartridge having the array of containers additionally comprising at least one container filled with storage buffer.

A further object of the present invention is to disclose a method of assaying an analyte in a water quality analyzer as defined in any of the above, which comprises further steps of obtaining and installing the cartridge according to in which the array of containers additionally comprises at least one container for organic buffer, the organic buffer optimized for use in assays of organic analytes.

A further object of the present invention is to disclose a method of assaying an analyte in a water quality analyzer as defined in any of the above, which comprises further steps of obtaining and installing the cartridge, in which the array of containers additionally comprising at least one container for metallic buffer, the metallic buffer optimised for use in assays of cationic heavy metals and metalloids analytes.

A further object of the present invention is to disclose a method of assaying an analyte in a water quality analyzer as defined in any of the above, which comprises further steps of obtaining and installing the cartridge in which the cartridge is adapted for storage in a module of the analyzer for predetermined periods of about one month.

A further object of the present invention is to disclose a method of assaying an analyte in a water quality analyzer as defined in any of the above, which comprises further steps of obtaining the cartridge as defined in any of the above, adapted for storage of in the module of the analyzer for a period of about 1-3 months.

A further object of the present invention is to disclose a method of assaying an analyte in a water quality analyzer as defined in any of the above, which comprises further steps of obtaining the cartridge and operating the analyzer so as to activate automated steps of transferring aliquots of the storage buffer into a predetermined number of the containers containing the freeze dried preparations of luminescent bacteria; incubating the freeze dried preparations of luminescent bacteria with the aliquots of the storage buffer so as to ensure hydration and biological activation of the bacteria; mixing the freeze dried preparations of luminescent bacteria with the aliquots of the storage buffer; adding organic or metal buffer to the assay chamber; adding test water to the assay chamber; mixing the water for about 2 seconds to homogenize the water and the buffers; adding the bacteria to the assay chamber; detecting the emitted light; and processing the results obtained, thereby assaying the analyte.

A further object of the present invention is to disclose a method of assaying an analyte in a water quality analyzer as defined in any of the above, which comprises further steps of obtaining and installing the disposable cartridge having the array additionally comprising at least one container containing disinfection buffer (DBC) mounted on the platform.

A further object of the present invention is to disclose a method of assaying an analyte in a water quality analyzer as defined in any of the above, which comprises further steps of obtaining and installing the cartridge having the array additionally comprising at least one container containing reference water (RWC) mounted on the platform.

A further object of the present invention is to disclose a method of assaying an analyte in a water quality analyzer as defined in any of the above, which comprises further steps of obtaining and installing the cartridge having the array additionally comprising a chamber for bacterial suspension.

A further object of the present invention is to disclose a method of assaying an analyte in a water quality analyzer as defined in any of the above, which comprises further steps of obtaining and installing the cartridge having any of the containers provided with volume sensors, the sensors provided with logical coupling means to a microprocessor in the analyzer.

A further object of the present invention is to disclose a method of assaying an analyte in a water quality analyzer as defined in any of the above, which comprises further steps of obtaining and installing the cartridge having the sensors adapted to signal data concerning contents of the containers and chambers to the microprocessor in the analyzer.

A further object of the present invention is to disclose a method of assaying an analyte in a water quality analyzer as defined in any of the above, which comprises further steps of obtaining and installing the cartridge having the platform adapted for traveling on a gantry, rail, lift, carriage or track.

A further object of the present invention is to disclose a method of assaying an analyte in a water quality analyzer as defined in any of the above, which comprises further steps of obtaining and installing the cartridge having at least one of the containers, chambers and fluid transferring means is adapted for manipulation by mechanical means.

A further object of the present invention is to disclose a method of assaying an analyte in a water quality analyzer as defined in any of the above, which comprises further steps of obtaining and installing the cartridge having the containers, chambers, and fluid transferring means attached by locking and mounting means to the platform at specific locations on the platform.

A further object of the present invention is to disclose a method of assaying an analyte in a water quality analyzer as defined in any of the above, which comprises further steps of obtaining and installing the cartridge having the platform adapted to lock into the analyzer in a predetermined configuration.

A further object of the present invention is to disclose a method of assaying an analyte in a water quality analyzer as defined in any of the above, which comprises further steps of obtaining and installing the cartridge having the platform adapted to be temperature controlled at specific container and chamber mounting locations, thereby holding the containers and chambers at predetermined temperatures.

A further object of the present invention is to disclose a method of assaying an analyte in a water quality analyzer as defined in any of the above, which comprises further steps of obtaining and installing the cartridge having the platform adapted to be cooled at specific container locations, and cooling the containers and chambers to predetermined temperatures.

A further object of the present invention is to disclose a method of assaying an analyte in a water quality analyzer as defined in any of the above, which comprises further steps of obtaining and installing the cartridge having fluid transferring means selected from a group consisting of inlet ports, outlet ports, syringes, valves, taps and connectors and transferring fluid via the fluid transferring means.

A further object of the present invention is to disclose a method of assaying an analyte in a water quality analyzer as defined in any of the above, which comprises further steps of obtaining and installing the cartridge having the array of containers additionally comprising at least one container filled with storage buffer.

Another object of the present invention is to disclose a method of assaying an analyte in a water quality analyzer as defined in any of the above, which comprises further steps of obtaining and installing the cartridge in which the array of containers additionally comprises at least one container at least one container for organic buffer, the organic buffer optimized for use in assays of organic analytes.

A further object of the present invention is to disclose a method of assaying an analyte in a water quality analyzer as defined in any of the above, which comprises further steps of obtaining and installing the cartridge in which the array of containers additionally comprises at least one container for metallic buffer, the metallic buffer optimised for use in assays of cationic heavy metals and metalloid analytes.

A further object of the present invention is to disclose a method of assaying an analyte in a water quality analyzer as defined in any of the above, which comprises further steps of obtaining and installing the cartridge, in which the cartridge is adapted for storage in a module of the analyzer for predetermined periods of about one month.

A further object of the present invention is to disclose a method of assaying an analyte in a water quality analyzer as defined in any of the above, which comprises further steps of obtaining the cartridge in which the cartridge is adapted for storage of in the module of the analyzer for a period of about 1-3 months.

A further object of the present invention is to disclose a method of assaying an analyte in a water quality analyzer as defined in any of the above, which comprises further steps of obtaining the cartridge in which the array of containers are provided with reagents and buffers adapted for use in assays selected from a group consisting of fluorometric assays, chemiluminescent assays and colourimetric assays.

A further object of the present invention is to disclose a disposable cartridge useful for preventing biofouling and biofilm build up and mishandling of reagents by manual operation of end user comprising a platform; an array of containers each separately containing reagents, buffers and freeze dried preparations of luminescent bacteria, the containers mounted on the platform; a fluid transferring means 70 for fluid transfer and mixing between the containers and the analyzer; and a plurality of connecting means adapted for connecting between the containers and the analyzer.

It is a core purpose of the invention to provide the cartridge adapted for conveyance to predetermined positions in said analyzer. The cartridge is further adapted for sequential manipulation of the containers and their contents such that the analyte can be assayed in the analyzer.

A further object of the present invention is to disclose the array comprising at least one container containing assay buffer, at least one container containing freeze dried reagent (FDC) and at least one assay chamber. The assay chamber is adapted for reacting the analyte, the reagents, the freeze dried suspensions of luminescent bacteria and the assay buffer together such that light detectable by said analyzer is emitted.

A further object of the present invention is to disclose the array additionally comprising at least one container containing disinfection buffer (DBC) mounted on said platform.

A further object of the present invention is to disclose the array additionally comprising at least one container containing reference water (RWC) mounted on the platform.

A further object of the present invention is to disclose the array additionally comprising a chamber for bacterial suspension.

A further object of the present invention is to disclose any of the containers provided with volume sensors. The sensors provided with logical coupling means to a microprocessor in the analyzer

A further object of the present invention is to disclose the sensors adapted to signal data concerning contents of the containers and the chambers to the microprocessor in the analyzer.

A further object of the present invention is to disclose the platform adapted for traveling on a gantry, rail, lift, carriage or track.

A further object of the present invention is to disclose at least one of containers, chambers and fluid transferring means adapted for manipulation by mechanical means.

A further object of the present invention is to disclose the containers, chambers, syringes and fluid transferring means attached by locking and mounting means to said platform at specific locations on the platform.

A further object of the present invention is to disclose the platform adapted to lock into the analyzer in a predetermined configuration.

A further object of the present invention is to disclose the platform adapted to be temperature controlled at specific container and chamber mounting locations, thereby holding the containers and chambers at predetermined temperatures.

A further object of the present invention is to disclose the platform adapted to be cooled at specific container locations, thereby cooling the containers and chambers to predetermined temperatures.

A further object of the present invention is to disclose the fluid transferring means selected from a group consisting of inlet ports, outlet ports, syringes, valves, taps and connectors.

A further object of the present invention is to disclose the array of containers additionally comprising at least one container filled with storage buffer.

A further object of the present invention is to disclose the array of containers additionally comprising at least one container at least one container for organic buffer. The organic buffer is optimized for use in assays of organic analytes.

A further object of the present invention is to disclose the array of containers additionally comprising at least one container for metallic buffer. The metallic buffer is optimized for use in assays of cationic heavy metals and metalloid analytes.

A further object of the present invention is to disclose the cartridge adapted for storage in a module of the analyzer for predetermined periods of about one month

A further object of the present invention is to disclose the cartridge adapted for storage of in the module of the analyzer for a period of about 1-3 months.

A further object of the present invention is to disclose the cartridge presented in kit form for assembly by the user according to instructions associated with the kit.

A further object of the present invention is to disclose the array of containers provided with reagents and buffers adapted for use in assays selected from a group consisting of fluorometric assays, chemiluminescent assays and colorimetric assays.

In a protocol for assaying an analyte in an automated water quality analyzer, a method for preventing biofouling and biofilm build up and mishandling of reagents by manual operation of the end user. The method comprises steps of (a) obtaining a disposable cartridge comprising a platform, an array of containers each separately containing reagents, buffers and freeze dried preparations of luminescent bacteria, a fluid transferring means adapted for fluid transfer and mixing between the containers and the analyzer, fluid transferring means adapted for connecting between the containers and the analyzer, the cartridge is adapted for conveying to predetermined positions in the analyzer, the cartridge further is adapted for sequential manipulation of at least some of said containers and their contents such that an analyte can be assayed in the analyzer; (b) installing the cartridge into the automated water quality monitoring analyzer; (c) operating the automatic analyzer thereby conveying the cartridge to the predetermined locations in the analyzer and sequentially manipulating the contents of the containers so as to activate the luminescent bacteria such that light is emitted; (d) detecting the emitted light; (e) processing the results obtained thereby assaying the analyte.

A further object of the present invention is to disclose the method comprising further steps of obtaining and installing the disposable cartridge having the array additionally comprising at least one container containing disinfection buffer (DBC) mounted on the platform.

A further object of the present invention is to disclose the method comprising further steps of obtaining and installing the cartridge having the array additionally comprising at least one container containing reference water (RWC) mounted on the platform.

A further object of the present invention is to disclose the method comprising further steps of obtaining and installing the cartridge having the array additionally comprising a chamber for bacterial suspension.

A further object of the present invention is to disclose the method comprising further steps of obtaining and installing the cartridge having any of the containers provided with volume sensors. The sensors are provided with logical coupling means to a microprocessor in the analyzer.

A further object of the present invention is to disclose the method comprising further steps of obtaining and installing the cartridge having the sensors adapted to signal data concerning contents of the containers and chambers to the microprocessor in the analyzer.

A further object of the present invention is to disclose the method further comprising steps of obtaining and installing the cartridge having the platform adapted for traveling on a gantry, rail, lift, carriage or track.

A further object of the present invention is to disclose the method further comprising steps of obtaining and installing the cartridge having at least one of the containers.

The chambers and the fluid transferring means are adapted for manipulation by mechanical means.

A further object of the present invention is to disclose the method further comprising steps of obtaining and installing the cartridge having the containers, the chambers, the syringes and the fluid transferring means attached by locking and mounting means to the platform at specific locations on the platform.

A further object of the present invention is to disclose the method further comprising steps of obtaining and installing the cartridge having the platform adapted to lock into said analyzer in a predetermined configuration

A further object of the present invention is to disclose the method further comprising steps of obtaining and installing the cartridge having the platform adapted to be temperature controlled at specific container and chamber mounting locations, thereby holding the containers and chambers at predetermined temperatures.

A further object of the present invention is to disclose the method further comprising steps of obtaining and installing the cartridge having the platform adapted to be cooled at specific container locations, and cooling said containers and chambers to predetermined temperatures.

A further object of the present invention is to disclose the method further comprising steps of obtaining and installing the cartridge having fluid transferring means selected from a group consisting of inlet ports, outlet ports, syringes, valves, taps and connectors and transferring fluid via said fluid transferring means.

A further object of the present invention is to disclose the method further comprising steps of obtaining and installing the cartridge having the array of containers additionally comprising at least one container filled with storage buffer

A further object of the present invention is to disclose the method comprising further steps of obtaining and installing the cartridge. The array of containers additionally comprises at least one container for organic buffer, said organic buffer optimized for use in assays of organic analytes.

A further object of the present invention is to disclose the method comprising further steps of obtaining and installing the cartridge having the array of containers additionally comprising at least one container for metallic buffer. The metallic buffer is optimized for use in assays of cationic heavy metals and metalloids analytes.

A further object of the present invention is to disclose the method further comprising steps of obtaining and installing the cartridge adapted for storage in a module of the analyzer for predetermined periods of about one month.

A further object of the present invention is to disclose the method further comprising steps of obtaining the cartridge adapted for storage of in the module of the analyzer for a period of about 1-3 months.

In a protocol for assaying an analyte in an automated water quality analyzer, the method comprises the steps of operating the analyzer so as to activate automated steps of transferring aliquots of the storage buffer into a predetermined number of the containers containing the freeze dried preparations of luminescent bacteria; incubating the freeze dried preparations of luminescent bacteria with the aliquots of the storage buffer so as to ensure hydration and biological activation of the bacteria; mixing the freeze dried preparations of luminescent bacteria with the aliquots of the storage buffer; adding organic or metal buffer to the assay chamber; adding test water to the assay chamber; mixing the water for about 2 seconds to homogenize the water and the buffers; adding the bacteria to the assay chamber; detecting the emitted light; and processing the results obtained, thereby assaying the analyte.

A further object of the present invention is to disclose the method for preventing biofouling build up and mishandling of reagents further comprising steps of obtaining and installing said disposable cartridge having the array additionally comprising at least one container containing disinfection buffer (DBC) mounted on the platform.

A further object of the present invention is to disclose the method for preventing biofouling build up and mishandling of reagents further comprising steps of obtaining and installing the cartridge having said array additionally comprising at least one container containing reference water (RWC) mounted on the platform.

A further object of the present invention is to disclose the method for preventing biofouling build up and mishandling of reagents further comprising steps of obtaining and installing the cartridge having the array additionally comprising a chamber for bacterial suspension.

A further object of the present invention is to disclose the method for preventing biofouling build up and mishandling of reagents further comprising steps of obtaining and installing the cartridge having any of the containers provided with volume sensors. The sensors are provided with logical coupling means to a microprocessor in the analyzer.

A further object of the present invention is to disclose the method for preventing biofouling build up and mishandling of reagents further comprising steps of obtaining and installing the cartridge having the sensors adapted to signal data concerning contents of the containers and chambers to said microprocessor in the analyzer.

A further object of the present invention is to disclose the method for preventing biofouling build up and mishandling of reagents further comprising steps of obtaining and installing the cartridge having the platform adapted for traveling on a gantry, rail, lift, carriage or track.

A further object of the present invention is to disclose the method for preventing biofouling build up and mishandling of reagents further comprising steps of obtaining and installing the cartridge having at least one of the containers, chambers and fluid transferring means is adapted for manipulation by mechanical means.

A further object of the present invention is to disclose the method for preventing biofouling build up and mishandling of reagents further comprising steps of obtaining and installing the cartridge having the containers, the chambers, the syringes and the fluid transferring means attached by locking and mounting means to the platform at specific locations on the platform.

A further object of the present invention is to disclose the method for preventing biofouling build up and mishandling of reagents further comprising steps of obtaining and installing the cartridge having the platform adapted to lock into the analyzer in a predetermined configuration.

A further object of the present invention is to disclose the method for preventing biofouling build up and mishandling of reagents further comprising steps of obtaining and installing the cartridge having the platform adapted to be temperature controlled at specific container and chamber mounting locations, thereby holding the containers and chambers at predetermined temperatures.

A further object of the present invention is to disclose the method for preventing biofouling build up and mishandling of reagents further comprising steps of obtaining and installing the cartridge having the platform adapted to be cooled at specific container locations, and cooling said containers and chambers to predetermined temperatures.

A further object of the present invention is to disclose the method for preventing biofouling build up and mishandling of reagents further comprising steps of obtaining and installing the cartridge having fluid transferring means selected from a group consisting of inlet ports, outlet ports, syringes, valves, taps and connectors and transferring fluid via said fluid transferring means.

A further object of the present invention is to disclose the method for preventing biofouling build up and mishandling of reagents further comprising steps of obtaining and installing the cartridge having the array of containers additionally comprising at least one container filled with storage buffer

A further object of the present invention is to disclose the method for preventing biofouling build up and mishandling of reagents further comprising steps of obtaining and installing the cartridge. The array of containers additionally comprises at least one container at least one container for organic buffer, said organic buffer optimised for use in assays of organic analytes.

A further object of the present invention is to disclose the method for preventing biofouling build up and mishandling of reagents further comprising steps of obtaining and installing the cartridge having said array of containers additionally comprising at least one container for metallic buffer optimized for use in assays of cationic heavy metals and metalloid analytes.

A further object of the present invention is to disclose the method for preventing biofouling build up and mishandling of reagents further comprising steps of obtaining and installing the cartridge adapted for storage in a module of the analyzer for predetermined periods of about one month.

A further object of the present invention is to disclose the method for preventing biofouling build up and mishandling of reagents further comprising steps of obtaining said cartridge adapted for storage of in the module of the analyzer for a period of about 1-3 months.

A further object of the present invention is to disclose the method for preventing biofouling build up and mishandling of reagents further comprising steps of obtaining said cartridge. The array of containers is provided with reagents and buffers adapted for use in assays selected from a group consisting of fluorometric assays, chemiluminescent assays and colourimetric assays.

### BRIEF DESCRIPTION OF THE FIGURES

FIG. 1 schematically shows a disposable cartridge 100 within an automated water quality monitoring analyzer 1000.

FIG. 2 schematically shows the automated water quality monitoring analyzer 1000 without the disposable cartridge.

FIG. 3 schematically shows the bacteria injection means 80

FIG. 4 schematically shows a top view of the disposable cartridge 100

FIG. 5 schematically shows a bottom view of the disposable cartridge 100

FIG. 6 schematically shows the assay moving means 40

FIG. 7 schematically shows an expanded view of the assay moving means 40

FIG. 8 schematically showing an expanded view of the assay chamber 30.

FIG. 9 schematically shows a Hardware Block Diagram.

FIG. 10 schematically shows a Software Block Diagram.

FIG. 11 schematically shows a detailed Software Block Diagram.

### DETAILED DESCRIPTION

In the following description, various aspects of the invention will be described. For the purposes of explanation, specific configurations and details are set forth in order to provide a thorough understanding of the invention. However, it will be also apparent to one skilled in the art that the invention may be practiced without specific details presented herein. Furthermore, well-known features may be omitted or simplified in order not to obscure the invention.

Many analyzers conventionally employed for continuously and automatically monitor various parameters of water quality are prone to generating false alarms for two main reasons:

Bio-fouling—prolonged exposure to water leads to build up of microorganisms along the tubes (biofilm) which leads to narrowing of their diameter and hence to deviations in the reagents' volumes dispensed into the reaction chamber. Once bio-fouling commences it is extremely difficult to eliminate. Eventually, tubes will get clogged and liquid flow will be stalled. The same process may occur inside the reaction chamber itself and lead to loss of transparency which is important when carrying out reactions that measure color change, fluorescence, or luminescence.

Users need to periodically install fresh batches of reagents inside the analyzer (in dedicated containers, flasks, etc). This process requires some care to correctly position the tubes leading from the containers into the measuring unit within the analyzer. Often, a certain preparatory procedure (such as hydration of freeze dried reagents) is required before installing the reagents inside the analyzer. Moreover, delay in proper refilling of reagents may lead to suction of air instead of liquid.

A main aspect of the present invention is to provide a disposable and sealed cartridge holding all reagents (in both freeze dried and liquid form), fluid transferring means for reagent dispensation, and reaction chambers. In some embodiments of the invention, volume sensors are installed in all containers within the cartridge. These sensors generate an alert when volumes of reagents are reaching their end. The containers could be either flasks, bottles, infusion bags, syringes.

In exemplary embodiments of the invention, in some containers within the cartridge there is provided specialized medium bacterial compositions in dry or liquid form, the compositions comprising about

| | |
|---|---|
| NaCl/KCl (%) | 1.0-3.0; |
| Inositol/lactose/trehalose/dextran (%) | 0.5-7.0; |
| Mg/Ca (mM) | 0.0-300; |
| Yeast Extract/Casamino acids(%) | 0.01-0.05; |
| BSA/egg albumin(%) | 0.02-1.0; and |
| Ethanol/methanol/propanol(%) | 0.1-3.5. |

In other exemplars of the invention there is provided a medium composition of

| | |
|---|---|
| NaCl/KCl (%) | 2.0-3.0; |
| Inositol/lactose/trehalose/dextran (%) | 5.0-7.0; |
| Mg/Ca (mM) | 1.0-200; |
| Yeast Extract/Casamino acids (%) | 0.01-0.05; |
| BSA/egg albumin(%) | 0.02-0.1; and |

Optionally, the medium composition comprises about:

| | |
|---|---|
| NaCl/KCl (%) | 2.0; |
| Inositol/lactose/trehalose/dextran (%) | 5.0; |
| Mg/Ca (mM) | 2.0; |
| Yeast Extract/Casamino acids (%) | 0.05; |
| BSA/egg albumin(%) | 0.05; and |
| Ethanol/methanol/propanol(%) | 1.5. |

The above compositions are recited and offered as non limited examples.

Optionally, the medium composition comprises at least one antibiotic preparation.

Optionally, the medium composition is provided as a liquid.

In an exemplary embodiment of the invention, there is provided a dehydrated bacterial suspension comprising a medium composition as described above and bacteria suspended therein.

In preferred embodiments of the invention the bacteria are dehydrated luminescent bacteria adapted for testing analytes in the water. In some exemplars of the invention, the disposable cartridge comprises at least one container containing tester bacteria of the GlnA mutant strain of E. coli (ET 12558) carrying the Lux-I deleted lux system of Vibrio fischeri. In some embodiments of the invention the disposable cartridge comprises at least one container containing tester bacteria of an E. coli nutrient-requiring mutant strain carrying the Lux-I deleted lux system of Vibrio fischeri. In some embodiments of the invention the disposable cartridge comprises at least one container containing tester bacteria of Vibrio harveyi. In some embodiments of the invention the disposable cartridge comprises at least one container containing tester bacteria of Vibrio fischeri. In yet another embodiment the reagents comprises tester bacteria of Photobacterium leiognathi. It is well within the scope of the present invention to provide the disposable cartridge with other bioluminescent tester bacteria or bioluminescent microbes selected according to the test being performed upon the water to be analyzed.

In some embodiments of the invention the aforementioned cartridge holds enough reagents for a month-long operation, after which it is disposed and replaced by a new one. Direct intervention by a human operator is thus minimized.

In a preferred embodiment of the invention the cartridge is controlled by a microprocessor within the fixed part of the analyzer.

Once the cartridge is installed, it is precisely locked on and positioned in a way that ensures tight contact between thermoelectric cooler units within the analyzer and the reaction chamber(s) (e.g., set to 30° C.) and the hydrated reagent container (e.g., set to 2° C.). The containers holding the assay buffers (pre-hooked to dispensers/syringes) are automatically juxtaposed on the conveyer belt as are the reaction chamber(s). Once the analyzer operation commences, the freeze dried reagent (e.g., luminescent bacteria) is hydrated and transferred into a specially cooled container/syringe; the reaction chamber(s) moves along the conveyer belt and sequentially collects the various components of the reaction, chosen, in a non limiting manner, from assay buffer, inlet water/reference water and hydrated reagent, or a combination thereof; it eventually reaches its final stop facing the reading device (e.g., photomultiplier). Once reading is recorded and reaction cycle is complete, the reaction chamber is tilted 180 degrees to spill its content. It is then washed with clean water and moved to its original starting position on the conveyer belt for another cycle. One of the containers within the cartridge holds a disinfection solution which periodically washes the reaction chamber to eliminate early stages of bio-film build up.

The use of a conveyer belt to bring the reaction chamber to the various reagents is a dramatic change from the common design in which multiple tubes linked to the various reagents containers feed the reaction chamber. This novel design reduces the number of tubes which are prone to clogging due to biofouling.

The preferred use of the disposable cartridge system is in the AquaVertiy-CCB (Continuous Contamination Biomonitor). This is an advanced automated online water quality monitoring analyzer that detects chemical contaminants in surface, ground, raw and treated drinking water, as well as any water reuse application with the need of water effluents surveillance. The test utilizes unique freeze-dried luminescent bacteria and proprietary assay buffer solutions to perform the automatic analysis. The analyzer provides alarms in the presence of very low concentrations of a wide spectrum of toxic agents including pesticides, herbicides, heavy metals, petroleum-based contaminants, protein synthesis inhibitors and respiratory inhibitors.

Batch analysis ensures long term and reliable data. The analyzer starts the automatic toxicity bioassay at defined interval times or by an external command giving out toxicity alarms only when detected and evaluated by its internal quality check procedures. The user may set the alarm to any chosen inhibition level. At the end of the analysis and after proper washing, the analyzer remains in standby mode, ready for a new cycle. The system is designed to periodically test negative (clean water) and positive (spiked toxic agent) controls.

The disinfection solution which periodically washes the reaction chamber was designed in such a way that when diluted (e.g., 1:10) and tested in the assay buffers it will generate a 50% inhibition in luminescence. Hence, one solution serves two purposes (disinfection and positive control).

Automatic flushing and cleaning cycles ensure long-term consistency of measurements and minimal biofouling build up.

A PC based Management Software communicating with the analyzer enables the operator to view and modify the main operation parameters; to directly examine real-time counts value; to obtain direct reading of the measurements collected by the unit; to program the analyzer in the monitor mode; to download the memory bank with measured data and to display the counts in graphical form. With the management software it is possible to manage all the operations normally required by the user during measurement. The software could be used either locally, with the PC directly connected to the analyzer or from remote locations via the internet.

The bacteria are provided in a freeze-dried state that ensures long term stability. Each of the proprietary bioassay reagent solutions is composed of a complex combination of chemicals specially designed to ensure both bacteria stability and maximal sensitivity to contaminants. An initial discrimination between organic and cationic heavy metals and metalloids contamination is provided by the two distinct assay buffers. The bioassay was found to be up to a hundred fold more sensitive than commonly used bioluminescence-based tests for a wide variety of tested toxic agents (Ulitzur et al. Environ Toxicology. 2002; 17(3):291-6).

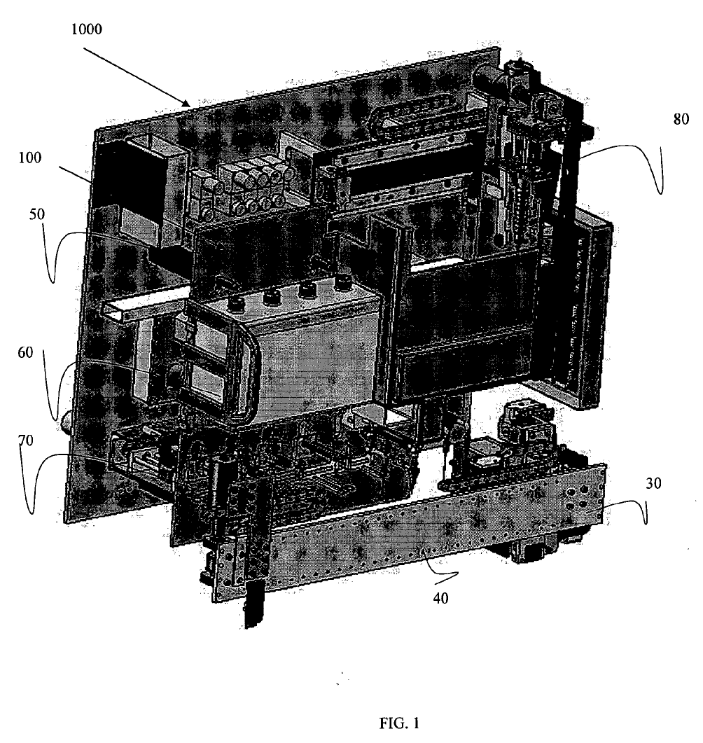

Reference is now made to FIG. 1, schematically showing a disposable cartridge 100 within an automated water quality monitoring analyzer 1000.

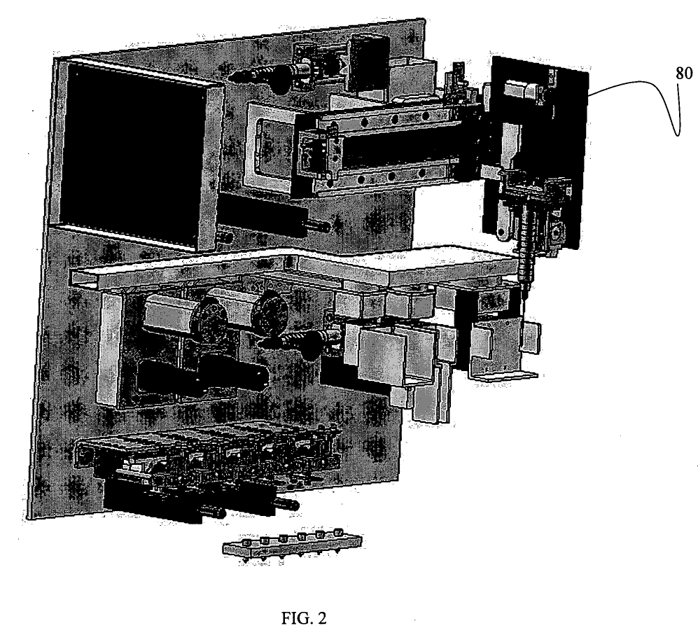

Reference is now made to FIG. 2, schematically showing the automated water quality monitoring analyzer 1000 without the disposable cartridge. A bacteria injection means 80, adapted for transferring freeze dried suspension of luminescent bacteria to storage container 25 is mounted on the analyzer.

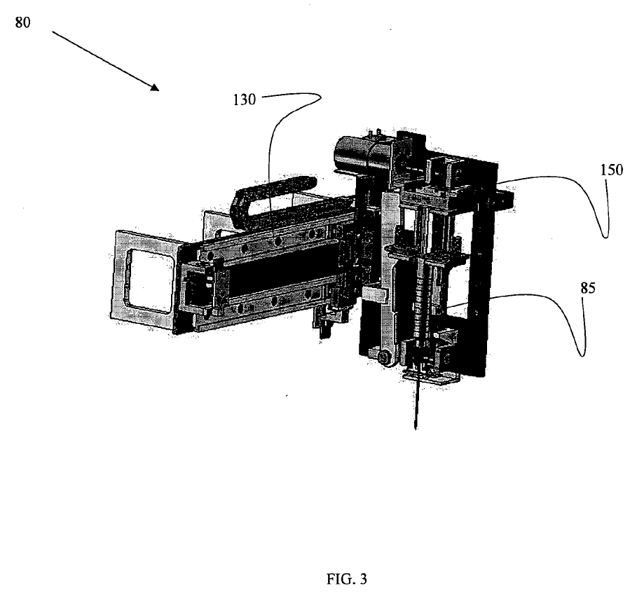

Reference is now made to FIG. 3, schematically showing the bacteria injection means 80, which comprises of at least one injecting means 85 (such as a syringe, an inlet port, an outlet port, pipettes, tubes, droppers, capillaries, pumps, valves, taps and connectors). The bacteria injecting means 80 is moveably mounted on the automated water quality monitoring analyzer 1000 via at least one horizontal actuator 130 (such as a conveyer belt, gantry, rail, lift, carriage or track) and an injection actuator 150. at least one vertical actuator 140 (such as a ratchet) can also be included. The injecting means 85 can thus automatically extract luminescent bacteria from a freeze dried suspension and transfer it to storage container 25.

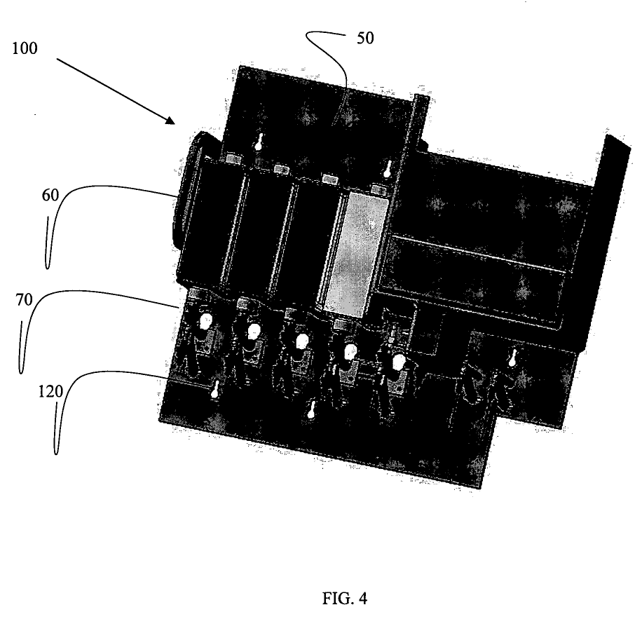

Reference is now made to FIG. 4, schematically showing a top view of the disposable cartridge 100, which comprises at least one container 60. The containers 60 are adapted for applying fluids, necessary for analyzing the analyte or sample 10. They can contain various materials, such as reagents, disinfection buffer (DBC), storage buffer, organic buffer (optimized for use in assays of organic analytes), metallic buffer (optimized for use in assays of cationic heavy metals and metalloid analytes), reference water (RWC), and any other material useful for interacting with the sample 10. The figure also shows at least one hooking means 120, useful for easily locking the platform 50 to the automated water quality monitoring analyzer 1000. Since the cartridge can be easily removed from the automated water quality monitoring analyzer 1000, either the containers 60 or the fluids within them can be easily refilled or changed. Fluid transferring means 70 (syringes, inlet ports, outlet ports, pipettes, tubes, droppers, capillaries, pumps, valves, taps or connectors) is coupled to the containers 60 and facilitates the transfer of the fluids to the assay chambers 30. The containers 60 can additionally have sensors. These sensors can sense the fluid volume in the containers, the content or temperature of fluid in the containers, or any other characteristics of the fluid, important for analysis. The data from the sensors can be provided with logical coupling means to a microprocessor in the automated water quality monitoring analyzer 1000.

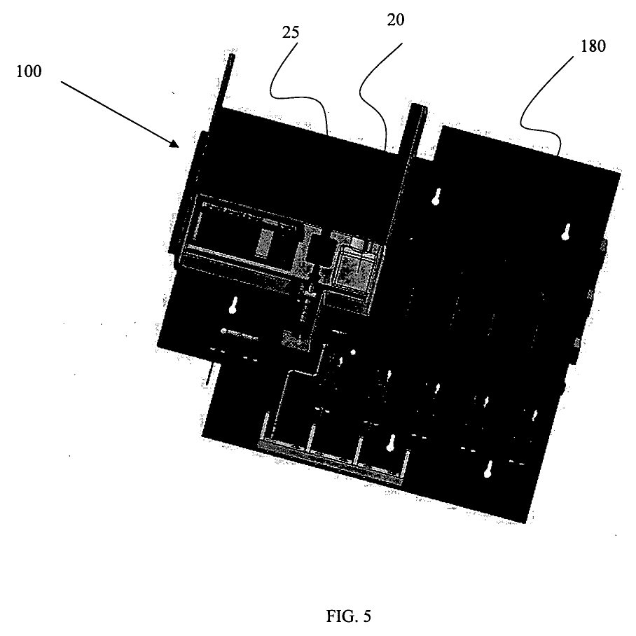

Reference is now made to FIG. 5, schematically showing a bottom view of the disposable cartridge 100, which is the part that faces the platform 50 which faces the automated water quality monitoring analyzer 1000. This view shows at least one freeze dried bacteria vial 20 and at least one storage container 25. Aliquots of freeze dried bacteria are dispensed into the storage container 25 by the bacteria injection means 80. This process can either be initiated automatically or manually. The figure also shows a hooking means 180, adapted for reversibly reliably and easily connecting the disposable cartridge 100 to the water quality monitoring analyzer 1000.

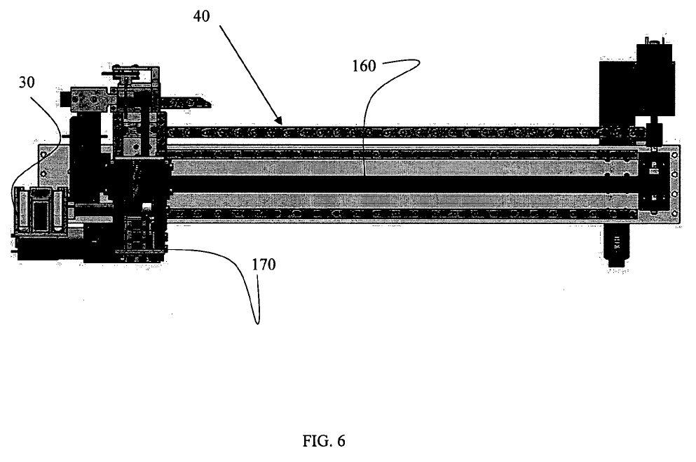

Reference is now made to FIG. 6, schematically showing the assay moving means 40, adapted for moving the assay chambers 30 along the fluid transferring means 70. The assay chambers 20 are moveably mounted on the assay moving means 40 via at least one horizontal actuator (such as a conveyer belt, gantry, rail, lift, carriage or track) 160, and at least one vertical actuator 170 (such as a ratchet). The assay moving means 40 is adapted for coupling the assay chambers 30 to the fluid transferring means 70 of the containers 60, so that the fluids in the containers 60 can be introduced into the assay chambers 30, thus performing the analysis. The movement can be controlled manually or automatically. In a preferred embodiment, the assay moving means is not a part of the disposable cartridge 100.

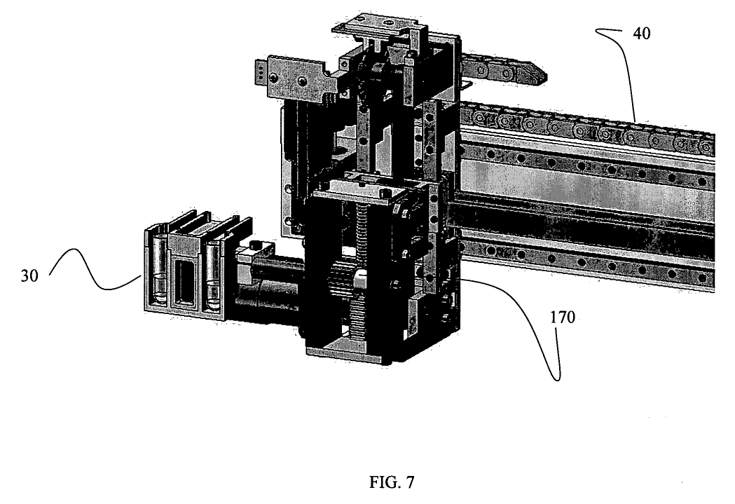

Reference is now made to FIG. 7, schematically showing an expanded view of the assay moving means 40, illustrating the vertical actuator 170.

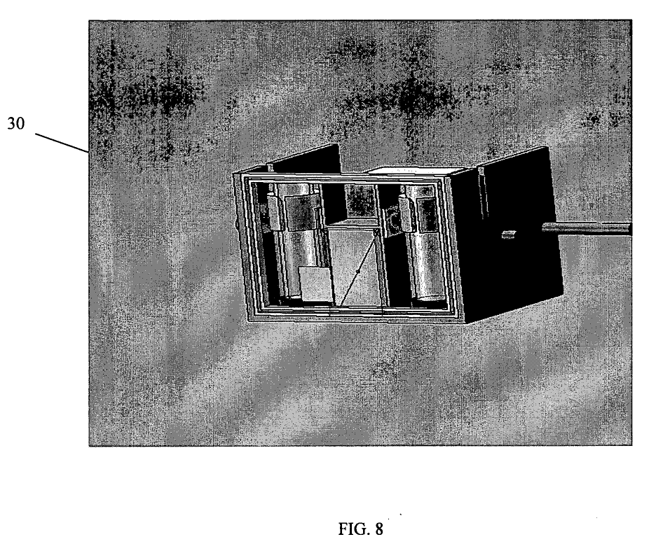

Reference is now made to FIG. 8, schematically showing an expanded view of the assay chamber 30.

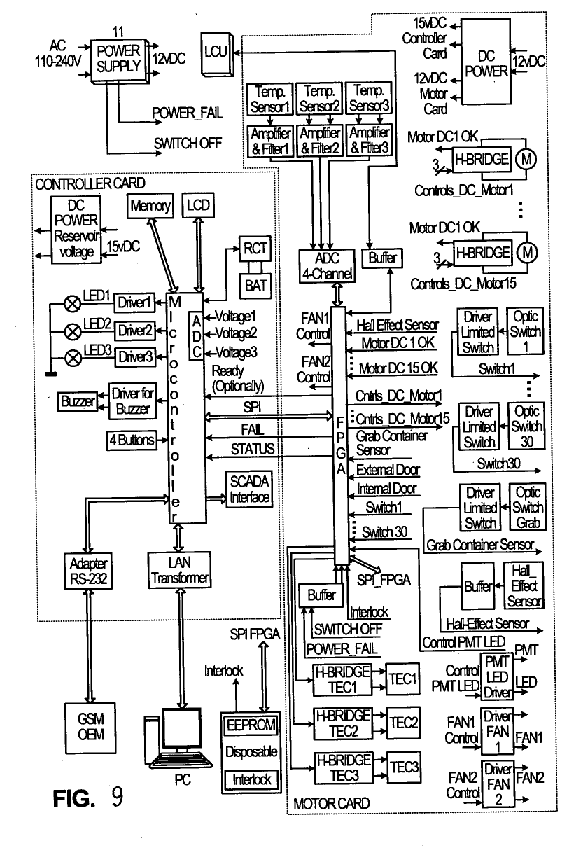

Reference is now made to FIG. 9, schematically showing a Hardware Block Diagram.
Reference is now made to

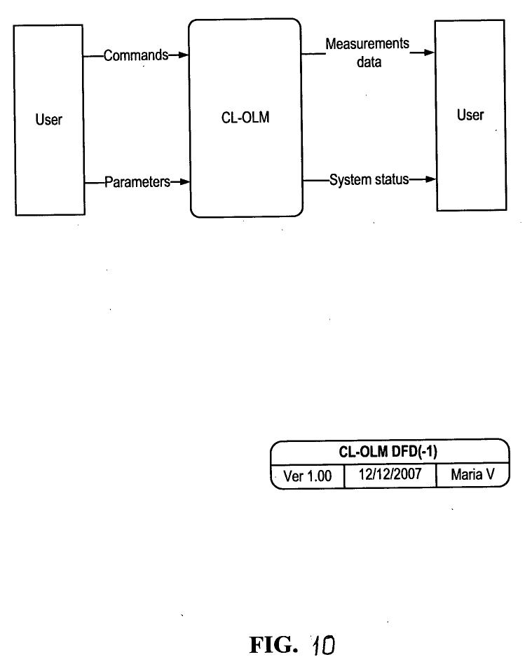

FIG. 10, schematically showing a Software Block Diagram.
Reference is now made to

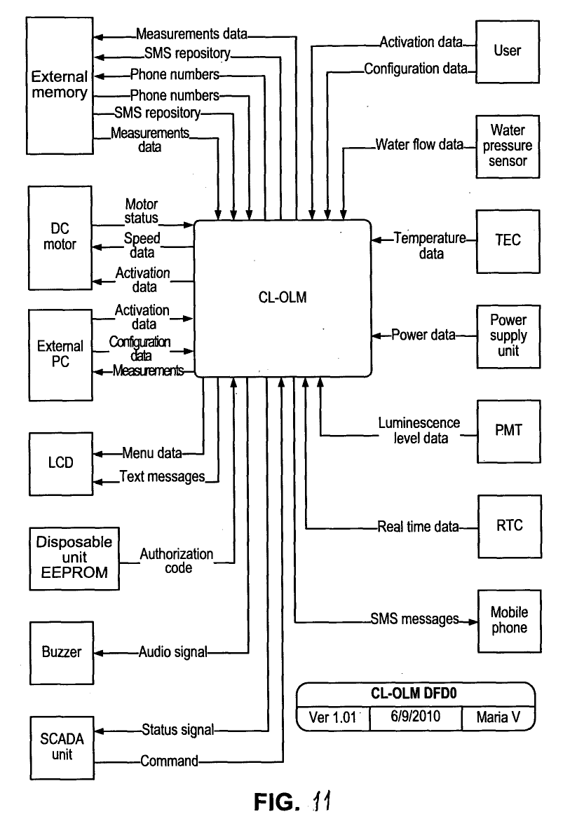

FIG. 11, schematically showing a detailed Software Block Diagram.

Reference is now made to a disposable cartridge 100 for use in assaying an analyte 10 in an automated water quality monitoring analyzer 1000. The aforementioned disposable cartridge 100 comprises
a. a platform 50
b. an array of containers 60 each separately containing reagents, buffers and freeze dried preparations of luminescent bacteria 20, which are mounted on the platform 50
c. a fluid transferring means 70 adapted for fluid transfer and mixing between the containers and the analyzer
d. a plurality of connecting means 120 adapted for connecting between the containers 60 and the analyzer,
wherein the cartridge 100 is adapted for conveyance to predetermined positions in the analyzer 1000, the cartridge 100 further adapted for sequential manipulation of the containers 60 and their contents such that the analyte 10 can be assayed in the analyzer 1000.

Reference is now made to the aforementioned cartridge 100 wherein the array comprises at least one container 60 containing assay buffer, at least one container containing freeze dried reagent (FDC) and at least one assay chamber, the assay chamber adapted for receiving the analyte, the reagents, the freeze dried suspensions of luminescent bacteria and the assay buffer together such that light detectable by the analyzer is emitted.

Reference is now made to the aforementioned cartridge wherein the array additionally comprises at least one container containing disinfection buffer (DBC) mounted on the platform.

Reference is now made to the aforementioned cartridge wherein the array additionally comprises at least one container containing reference water (RWC) mounted on the platform.

Reference is now made to the aforementioned cartridge wherein the array additionally comprises a chamber for bacterial suspension.

Reference is now made to the aforementioned cartridge wherein any of the containers are provided with volume sensors, which are provided with logical coupling means to a microprocessor in the analyzer.

Reference is now made to the aforementioned cartridge wherein the sensors are adapted to signal data concerning contents of the containers and chambers to a microprocessor in the analyzer.

Reference is now made to the aforementioned cartridge wherein the platform is adapted for traveling on a gantry, rail, lift, carriage or track.

Reference is now made to the aforementioned cartridge wherein at least one of the containers, chambers and fluid transferring means is adapted for manipulation by mechanical means.

Reference is now made to the aforementioned cartridge wherein the containers, chambers, syringes and fluid transferring means are attached by locking and mounting means to the platform at specific locations on the platform.

Reference is now made to the aforementioned cartridge wherein the platform is adapted to lock into the analyzer in a predetermined configuration.

Reference is now made to the aforementioned cartridge wherein the platform is adapted to be temperature controlled at specific container and chamber mounting locations, thereby holding the containers and chambers at predetermined temperatures.

Reference is now made to the aforementioned cartridge wherein the platform is adapted to be cooled at specific container locations, thereby cooling the containers and chambers to predetermined temperatures.

Reference is now made to the aforementioned cartridge wherein the fluid transferring means are selected from a group consisting of inlet ports, outlet ports, syringes, valves, taps and connectors.

Reference is now made to the aforementioned cartridge wherein the array of containers additionally comprises at least one container filled with storage buffer

Reference is now made to the aforementioned cartridge wherein the array of containers additionally comprises at least one container for organic buffer, which is optimised for use in assays of organic analytes

Reference is now made to the aforementioned cartridge wherein the array of containers additionally comprises at least one container for metallic buffer, which is optimized for use in assays of cationic heavy metals and metalloid analytes.

Reference is now made to the aforementioned cartridge wherein the cartridge is adapted for storage in a module of the analyzer for predetermined periods of about one month

Reference is now made to the aforementioned cartridge wherein the cartridge is adapted for storage of in the module of the analyzer for a period of about 1-3 months.

Reference is now made to the aforementioned cartridge wherein the cartridge is presented in kit form for assembly by the user according to instructions associated with the kit.

Reference is now made to a method of assaying an analyte in a water quality analyzer wherein the method comprises steps of
a. obtaining a disposable cartridge comprising
   i. a platform 50
   ii. an array of containers 60 each separately containing reagents, buffers and freeze dried preparations of luminescent bacteria 20
   iii. a fluid transferring means 70 adapted for fluid transfer and mixing between the containers 60 and the analyzer 1000
   iv. fluid transferring means adapted for connecting between the containers and the analyzer,
      wherein the cartridge is adapted for conveying to predetermined positions in the analyzer, the cartridge further adapted for sequential manipulation of at least some of the containers and their contents such that an analyte can be assayed in the analyzer
b. installing the cartridge 100 into the automated water quality monitoring analyzer 1000
c. operating the automatic analyzer 1000 thereby conveying the cartridge 100 to a predetermined locations in the analyzer and sequentially manipulating the contents of the containers so as to activate the luminescent bacteria such that light is emitted
d. detecting emitted light and
e. processing the results obtained thereby assaying the analyte.

Reference is now made to a method of assaying an analyte in a water quality analyzer as aforementioned wherein the method comprises further steps of obtaining and installing a disposable cartridge having an array additionally comprising at least one container containing disinfection buffer (DBC) mounted on a platform

Reference is now made to a method of assaying an analyte in a water quality analyzer as aforementioned wherein the method comprises further steps of obtaining and installing the cartridge having and array additionally comprising at least one container containing reference water (RWC) mounted on a platform

Reference is now made to the aforementioned method of assaying an analyte in a water quality analyzer wherein the method comprises further steps of obtaining and installing a cartridge having an array additionally comprising a chamber for bacterial suspension.

Reference is now made to the aforementioned method of assaying an analyte in a water quality analyzer wherein the method comprises further steps of obtaining and installing a cartridge having any of the containers provided with volume sensors, which are provided with logical coupling means to a microprocessor in the analyzer

Reference is now made to the aforementioned method of assaying an analyte in a water quality analyzer wherein the method comprises further steps of obtaining and installing a cartridge having sensors adapted to signal data concerning contents of the containers and chambers to a microprocessor in the analyzer.

Reference is now made to the aforementioned method of assaying an analyte in a water quality analyzer wherein the method comprises further steps of obtaining and installing a cartridge having a platform adapted for traveling on a gantry, rail, lift, carriage or track.

Reference is now made to the aforementioned method of assaying an analyte in a water quality analyzer wherein the method comprises further steps of obtaining and installing a cartridge having at least one of the containers, chambers and fluid transferring means is adapted for manipulation by mechanical means.

Reference is now made to the aforementioned method of assaying an analyte in a water quality analyzer wherein the method comprises further steps of obtaining and installing a cartridge having containers, chambers, syringes and fluid transferring means attached by locking and mounting means to a platform at specific locations on the platform.

Reference is now made to the aforementioned method of assaying an analyte in a water quality analyzer wherein the method comprises further steps of obtaining and installing a cartridge having a platform adapted to lock into the analyzer in a predetermined configuration

Reference is now made to the aforementioned method of assaying an analyte in a water quality analyzer wherein the method comprises further steps of obtaining and installing a cartridge having a platform adapted to be temperature controlled at specific container and chamber mounting locations, thereby holding the containers and chambers at predetermined temperatures.

Reference is now made to the aforementioned method of assaying an analyte in a water quality analyzer wherein the method comprises further steps of obtaining and installing a cartridge having a platform adapted to be cooled at specific container locations, and cooling the containers and chambers to predetermined temperatures.

Reference is now made to the aforementioned method of assaying an analyte in a water quality analyzer wherein the method comprises further steps of obtaining and installing a cartridge having fluid transferring means selected from a group consisting of inlet ports, outlet ports, syringes, valves, taps and connectors and transferring fluid via fluid transferring means.

Reference is now made to the aforementioned method of assaying an analyte in a water quality analyzer wherein the method comprises further steps of obtaining and installing a cartridge having an array of containers additionally comprising at least one container filled with storage buffer.

Reference is now made to the aforementioned method of assaying an analyte in a water quality analyzer wherein the method comprises further steps of obtaining and installing a cartridge wherein an array of containers additionally comprises at least one container for organic buffer, which is optimised for use in assays of organic analytes.

Reference is now made to the aforementioned method of assaying an analyte in a water quality analyzer wherein the method comprises further steps of obtaining and installing a cartridge having an array of containers additionally comprising at least one container for metallic buffer, which is optimised for use in assays of cationic heavy metals and metalloid analytes

Reference is now made to the aforementioned method of assaying an analyte in a water quality analyzer wherein the method comprises further steps of obtaining and installing a cartridge, which is adapted for storage in a module of the analyzer for predetermined periods of about one month.

Reference is now made to the aforementioned method of assaying an analyte in a water quality analyzer wherein the method comprises further steps of obtaining a cartridge and adapting the cartridge for storage in a module of the analyzer for a period of about 1-3 months.

Reference is now made to the aforementioned method wherein the method comprises further steps of obtaining a cartridge and operating the analyzer so as to activate automated steps of
1. transferring aliquots of storage buffer into a predetermined number of containers containing freeze dried preparations of luminescent bacteria
2. incubating the freeze dried preparations of luminescent bacteria with aliquots of storage buffer so as to ensure hydration and biological activation of the bacteria
3. mixing the freeze dried preparations of luminescent bacteria with aliquots of storage buffer
4. adding organic or metal buffer to an assay chamber
5. adding test water to the assay chamber, the tested water having gone through a tube that is heated/cooled to the chosen optimal assay chamber (e.g., 30° C.) so that once it reaches the assay chamber it is already at the right temp for obtaining fast and optimal results
6. mixing the water for about 2 seconds to homogenize the water and the buffers
7. adding the bacteria to the assay chamber
8. incubating mixture for a predetermined time
9. detecting emitted light every minute for 15 minutes
10. processing the results obtained, thereby assaying the analyte.

Reference is now made to the aforementioned method of assaying an analyte in a water quality analyzer wherein the method comprises further steps of obtaining and installing the disposable cartridge having an array additionally comprising at least one container containing disinfection buffer (DBC) mounted on the platform.

Reference is now made to the aforementioned method of assaying an analyte in a water quality analyzer wherein the method comprises further steps of obtaining and installing the cartridge having an array additionally comprising at least one container containing reference water (RWC) mounted on the platform.

Reference is now made to the aforementioned method of assaying an analyte in a water quality analyzer wherein the method comprises further steps of obtaining and installing a cartridge having an array additionally comprising a chamber for bacterial suspension.

Reference is now made to the aforementioned method of assaying an analyte in a water quality analyzer wherein the method comprises further steps of obtaining and installing a cartridge having any of the containers provided with volume sensors, which are provided with logical coupling means to a microprocessor in the analyzer.

Reference is now made to the aforementioned method of assaying an analyte in a water quality analyzer wherein the method comprises further steps of obtaining and installing a cartridge having sensors adapted to signal data concerning contents of the containers and chambers to a microprocessor in the analyzer.

Reference is now made to the aforementioned method of assaying an analyte in a water quality analyzer wherein the method comprises further steps of obtaining and installing a cartridge having at least one of the containers, chambers and fluid transferring means is adapted for manipulation by mechanical means.

Reference is now made to the aforementioned method of assaying an analyte in a water quality analyzer wherein the method comprises further steps of obtaining and installing a cartridge having containers, chambers, syringes and fluid transferring means attached by locking and mounting means to a platform at specific locations on the platform.

Reference is now made to the aforementioned method of assaying an analyte in a water quality analyzer wherein the method comprises further steps of obtaining and installing a cartridge having a platform adapted to lock into the analyzer in a predetermined configuration.

Reference is now made to the aforementioned method of assaying an analyte in a water quality analyzer wherein the method comprises further steps of obtaining and installing a cartridge having a platform adapted to be temperature controlled at specific container and chamber mounting locations, thereby holding the containers and chambers at predetermined temperatures.

Reference is now made to the aforementioned method of assaying an analyte in a water quality analyzer wherein the method comprises further steps of obtaining and installing the cartridge having the platform adapted to be cooled at specific container locations, and cooling the containers and chambers to predetermined temperatures.

Reference is now made to the aforementioned method of assaying an analyte in a water quality analyzer wherein the method comprises further steps of obtaining and installing a cartridge having fluid transferring means selected from a group consisting of inlet ports, outlet ports, syringes, valves, taps and connectors and transferring fluid via fluid transferring means.

Reference is now made to the aforementioned method of assaying an analyte in a water quality analyzer wherein the method comprises further steps of obtaining and installing a cartridge having an array of containers additionally comprising at least one container filled with storage buffer.

Reference is now made to the aforementioned method of assaying an analyte in a water quality analyzer wherein the method comprises further steps of obtaining and installing a cartridge wherein an array of containers additionally comprises at least one container for organic buffer, which is optimised for use in assays of organic analytes.

Reference is now made to the aforementioned method of assaying an analyte in a water quality analyzer wherein the method comprises further steps of obtaining and installing a cartridge having an array of containers additionally comprising at least one container for metallic buffer, which is optimised for use in assays of cationic heavy metals and metalloid analytes.

Reference is now made to the aforementioned method of assaying an analyte in a water quality analyzer wherein the method comprises further steps of obtaining and installing a cartridge adapted for storage of said reagents in a module of the analyzer for predetermined periods of about one month.

Reference is now made to the aforementioned method of assaying an analyte in a water quality analyzer wherein the method comprises further steps of obtaining a cartridge which is adapted for storage of said reagents in a module of the analyzer for a period of about 1-3 months.

### Example

Below is a description of an automated water quality monitoring analyzer which utilises the disposable cartridges described herein. It is herein acknowledged that the analyzer is described as an example, and that it will occur to a person skilled in the art that there are other configurations of water quality monitoring analyzers to which the disposable cartridges of the invention are compatible, suitable and useful. Such disposable cartridges are to be considered well within the scope of the invention.

Glossary of Terms Used in this Example:

| Term | Description |
|---|---|
| BIT | Built In Test |
| DFD | Data Flow Diagram |
| EEPROM | Electrically Erasable Programmable Read-Only Memory |
| GSM | Global System for Mobile |
| GUI | Graphical User Interface |
| IP | Internet Protocol |
| LAN | Local Area Network |
| LCD | Liquid Crystal Display |
| LED | Light Emitting Diode |
| PC | Personal Computer |
| PMT | Photomultiplier tube |
| SCADA | Supervisory Control and data Acquisition |
| SMS | Short Message Service |
| SW | Software |
| TBD | To Be Defined |
| TEC | Thermo-electric cooler |

### CheckLight Automated Water Quality Monitoring Analyzer

### System Overview

The following describes an automatic analyzer for early warning of chemical contamination in water. It is in the form of a manual which is incorporated in it's entirety by reference, yet is exemplary. Other versions of the device are indeed contemplated. The device uses a renewable suspension of luminescent bacteria that produces light as a respiratory by-product. The renewable suspensions, buffers and reagents are all contained within components of the disposable cartridges of the invention. When these bacteria are automatically mixed with a water sample, their light production, which is directly tied to critical metabolic cellular pathways, is decreased in proportion to the toxicity (concentration of chemicals) in the sample. The analyzer provides alarms in the presence of low concentrations of a wide spectrum of toxic contaminants.

### Document Purpose

This document describes the software requirements of the AquaVerity-CCB software.

### 1. Requirements

### 1.1. Required States and Modes

State 1—Initialization state

The software shall perform all peripherals initialization and initial washing

State 2—Halt state

While disposable cartridge is not connected, the software shall perform periodic tests and respond to user inputs

State 3—Regular activity

Once the disposable cartridge is connected the toxicity test cycles and system washing shall be performed constantly.

State 4—Alarm state
See Feature F008—System monitoring

### 1.2. Functional Requirements

Feature F001—Mechanical interface
Description: The following substance containers are present in the system:
1. 4 freeze-dried bacteria vials
2. 1 storage buffer, which contains solution for bacteria hydration & storage
3. Bacteria chamber, which stores the bacteria after it was mixed with storage buffer solution
4. Metal buffer
5. Organic buffer
6. Disinfection solution
7. 2 assay chambers
8. Grab sample container, in which two different contaminated water samples can be stored
9. Wash container
10. Reference water container
11. Inlet water—the water from the tested source
   REQ1_01: The software shall be interfaced to DC motor
   REQ1_02: The software shall draw predefined quantity of content from one container to another
   REQ1_03: The software shall provide the exact defined quantity of container's content

| Solution | Rate |
|---|---|
| Water | 0.8 ml |
| Bacteria suspension | 0.01 ml |
| Organic buffer | 0.2 ml |
| Metal buffer | 0.2 ml |

REQ1_04: The software shall calculate and provide the remaining volume of contents in storage buffer and bacteria vials

| Name | Quantity | Quantity in ml |
|---|---|---|
| Freeze dried bacteria | 4 | 6 ml after hydration |
| | | with storage buffer |
| Storage buffer | 1 | 80 ml total |
| Bacteria chamber | 4 | 15 ml |
| Organic buffer | 1 | 650 ml |
| Metal buffer | 1 | 650 ml |
| Disinfection solution | 1 | 650 ml |

REQ1_05: The software shall monitor the state of water flow sensor
REQ1_06: The software shall recognize disposable unit connection
REQ1_07: The software shall mix bacteria chamber with air bubbles before test start
Feature F002_Data storage and extraction

### Description

REQ2_01: All luminescence level data measurement results and system status shall be stored for a period of 32 days

### REQ2_02: Canceled

REQ2_03: The software shall store repository of SMS text messages and telephone numbers
REQ2_03_01:7 SMS messages shall be defined in text file [provided by user (see

### REQ8_03)

REQ2_03_02: Maximal length of SMS message shall be 40
REQ2_03_03: 5 phone numbers shall be defined in system
REQ2_03_04: Length of phone number shall be 18

REQ2_04: The software shall store the records and descriptions of system malfunctions, timings of occurrence and repairing (if applicable). All activities of “reset alarm” shall be listed in a dedicated file in the memory, to be downloaded with every status report. All activities of “Toxicity alarms” shall be listed in a dedicated file in the memory, to be downloaded with every status report

REQ2_05: The software shall erase all data upon disposable unit replacement. The software shall notify the user that all data would be erased after new disposable is entered. The PC software shall provide the possibility for data export.
REQ2_06: The software shall send the measurement to host computer (if not stand alone) periodically for backup (see REQ3_05_05) and graphical representation purposes
REQ2_06_01: The graphical measurements representation shall include last 15 test cycles “representative measurement”. Representative measurement shall be the last measurement in test cycle regardless whether the test was finished in time or early (in case of alarm). The same definition applies to the textual measurements representation on LCD.
REQ2_06_02: In graphical representation—if the measurement exceeds the limits of toxicity—it shall be emphasized on the screen with different color
REQ2_07: The software shall enable the extraction of data at any time upon user request through communication systems
Feature F003_User interface

### Description

REQ3_01: The software shall display textual data on the LCD and textual and graphic data via generic web browser. The data displayed on the screens shall include:
1. Configuration—parameters insertion. Parameters in system are as follows:
   a. Luminescence level ascent and descent percentage for toxicity alarm generation
   b. Time allowed from the moment of temperature off limits warning generation till alarm generation
   c. Reaction time (sample, reference, positive control)
   d. Temperature limits (bacteria chamber and assay chamber)
   e. Disinfection cycle
   f. Reference reading cycle (regular and for the first 24 hours)
   g. Positive control cycle
   h. Background reference reading
   i. Washing rate parameter
   j. Initial light reading level
   k. Phone numbers
2. Alarms, messages (see system monitoring)
3. Real time (date, hour, minutes)
4. 15 last readings of PMT (for each assay chamber) and indication whether it is a reference or sample run.
5. Current temperatures (bacteria and assay chambers)
   REQ3_02: The software shall indicate current system's state by controlled light indicators (LEDs and browser):
1. Green light—the system functionalizes properly, no water contamination
2. Red light—the water is contaminated
3. Yellow light—system malfunctions
   REQ3_03: The software shall recognize physical button pressure and act according to pressed button definition. Number of buttons in system is 4:
1. “Enter”
2. “ESC”—back to previous screen
3. “+”—cycle character/digit rolling
4. “−”—cycle rolling between lines
   REQ3_04: The software shall manage local audible alert (buzzer)
   REQ3_04_01: The software shall activate buzzer in case of alarm in system
   REQ3_04_02: The software shall allow user to disable and enable buzzer
   REQ3_05: The software shall provide user interface on the host PC, through Web Bowser via Ethernet connection
   REQ3_05_01: Local IP addresses management should support both dynamic and static work mode.
   REQ3_05_02: The software shall store historical data of both collected data and device status in Excel (.CSV) format.
   REQ3_05_03: The software shall collect, analyze measurements information collected from AQUAVERITY-CCB device. The software shall present a graphical representation of the last 15 measurements (end-point) collected from AQUAVERITY-CCB device (see REQ2_06_01).
   REQ3_05_04: The software shall enable SW upgrade to AQUAVERITY-CCB devices
   REQ3_05_05: The software shall send email messages to one e-mail address defined as a parameter in system
   REQ3_05_05_01: The software shall send a mail message once a day containing all measurements collected since the last mail sent (backup). Time of sending mail message shall be a parameter configured by technician or admin
   REQ3_05_05_02: Mail address shall be a parameter configured by technician or admin
   REQ3_05_05_03: All SMS messages defined in system shall be sent in e-mail also.
   REQ3_05_06: The software shall enable SMS messages file upgrade (by technician authority only)
   REQ3_05_07: The software shall enable default parameters file upgrade (by technician authority only)
   Feature F004—Temperature control
   Description: The system includes 3 TEC units that are software controlled
   REQ4_01: The software shall activate TECs units
   REQ4_02: The software shall read the values from 3 temperature sensors, placed on the TEC units
   REQ4_03: The software shall keep the temperature inside assay chambers within the defined range (parameter)
   REQ4_04: The software shall keep the temperature inside bacteria chamber within the defined range (parameter)
   REQ4_05: The software shall keep the temperature inside bacteria vials within the defined range (parameter)
   REQ4_06: The software shall keep the temperature inside storage buffer container within the defined range (parameter)
   Feature F005—Power supply unit

### Description

REQ5_01: The software shall monitor the following power supplies:
1. Voltage 1—12 V
2. Voltage 2—3.3 V
3. Voltage 3—5 V
   REQ5_02: Recognition: The software shall be able to identify the power shutdown and power fail
   REQ5_02_01: Power failure recognition with uninterruptible power supply in system: Upon receiving “Power supply failure” signal the software shall turn off all TEC units with delay of one second between and set a timeout of 1 minute. After 1 minute the software shall check the power supply status. If power supply is back to normal—the software shall continue its activity. Otherwise—power supply failure recognized.
   REQ5_03: The software shall perform 5 minutes delay after power return, following absence of power
   REQ5_04: Recovery: If power is absent less than 1 minute—the software shall continue interrupted activity. Otherwise, the software shall initialize its activity from the beginning, like after power up

Temperature of bacteria chamber shall be measured. If the temperature exceeds maximum value defined for bacteria chamber—the software shall generate appropriate warning. Following activities—the software shall stop

REQ5_05: The software shall store the exact date and time of power shortage in external memory sector designated exactly for this purpose. The pointer to the current flash memory address in measurements file shall be stored in the same sector. Those actions shall be performed within about 1 seconds time.
REQ5_06: The software shall notify the user upon power shortage detection and upon returning to normal activity afterwards via GSM
Feature F006—Real time clock

### Description

REQ6_01: The software shall enable updating the real time clock
REQ6_02: The software shall enable reading time and date from the real time clock
Feature F007—PMT unit management

### Description

REQ7_01: The software shall read the luminescence level data from the PMT unit
REQ7_02: The software shall recognize PMT saturation
REQ7_03: The software shall manage 1 small LED, designated for PMT functionality test
Feature F008—System monitoring

### Description

REQ8_01: The software shall perform power up BIT and periodic BIT
REQ8_01_01: The software shall enable LEDs testing by pressing two buttons (TBD) together
REQ8_02: The software shall alarm the user in different channels:
1. LEDs
2. Textual message on the screen
3. SMS message
4. Audio signal
   REQ8_03: The software shall send SMS message in following cases:
1. Toxicity alert—metalic
2. Toxicity alert—organic
3. Instrument malfunction
4. Disposable needs replacement
5. Power failure
6. Power failure recovery
7. Water flow problem
   REQ8_04: The software shall generate warning in the following cases:
1. Buffers and bacteria volume are close to low level—message on the screen and/or on the display on host computer, if connected
   a. Metal container-when volume reaches 10 ml
   b. Organic container—when volume reaches 10 ml
   c. Bacteria container—when volume reaches 1 ml
   d. Disinfection container—when volume reaches 20 ml.
   e. Reference external container—when volume reaches 100 ml.
   f. Storage buffer container—when volume reaches 15 ml
2. Temperature off limits (storage buffer, bacteria chamber and assay chambers)—message on the screen and/or on the display on host computer, if connected
3. Power return—via SMS message

The warning is not an alarm. The software shall continue its activity as usual.

REQ8_05: The software shall generate alarm in the following cases:
1. Power supply failure
2. No inlet water flow
3. No reference water flow
4. Water outlet blocked
5. Temperature off limits (both bacteria chamber and assay chamber) for a predefined period of time—parameter
6. Abnormal change (ascend or descend) in light level in reference water (relative and/or absolute level)-parameter
7. Initial reference water light level is too low—parameter
8. Toxicity alert (metallic or organic)
9. Positive control not working
10. Buffers and bacteria volume reaching low level
11. PMT malfunction/saturation
12. Disposable cartridge hasn't been replaced on time
13. Un-authorized disposable unit insertion attempt
14. Motor failure—indication and identification of the relevant component
15. Microcontroller's failure
   REQ8_06: System behavior in alarm state:
1. Temperature off limits—the software shall proceed for a predefined period of time (parameter). If temperature remains out of limits, the software shall stop
2. Motor failure—the software shall stop
3. Microcontroller's failure—the software shall stop
4. Disposable not replaced—the software shall stop
5. Un-authorized disposable unit connected—the software shall stop
6. PMT failure—the software shall stop
7. Toxicity alert—the software shall continue reading cycles
8. Reagents low volume—the software shall continue 6×15 min (=2 hrs) cycles and then stop
9. Positive control fail—the software shall rerun positive control cycle and if repeated then stop
10. No inlet water flow—the software shall stop
11. No reference water flow—the software shall stop
12. Water outlet blocked—the software shall stop
13. Reference reading drops sharply from last reading—the software shall rerun reference reading and if repeated then stop
14. Initial low reading of light after inserting a new cartridge (below x relative light units—parameter)—the software shall stop
15. Power supply failure—see REQ5_04
   Feature F009—Toxicity test cycle

### Description

### REQ9_01: Preparations:

1. Transferring the solution (5 ml) from storage buffer into freeze-dried bacteria vials
   2. Transferring the solution from freeze-dried bacteria into bacteria chamber (the hydration of freeze dried bacteria shall be performed 24 hours before usage, excluding the first vial, which will be used after system stabilization—TBD)
3. Mixing the solution with bacteria
4. Adding organic/metal buffer to assay chamber
5. Adding water to assay chamber (test cell), mixing water for 2 seconds.
6. Adding bacteria to assay chamber—bacteria shall not be added before the homogeneity of the water and the buffer is ensured. PMT reading shall not commence before proper mixing of bacteria is ensured
   REQ9_02: Test period is defined to be X minutes. The parameter shall be defined by user (see NFREQ18_02) Organic toxicity test and metal toxicity test shall have separate parameters. The luminescence level measurement shall be taken every minute.
   REQ9_03: The background reference measurement shall be taken once in a while (parameter, see NFREQ18_02). The luminescence level shall be measured when assay chambers are empty. This offset shall be subtracted from all measurements.
   REQ9_04: 3 types of test cycle shall be defined:
   REQ9_04_01: Positive control cycle

The software shall perform a positive control test using disinfection solution (diluted 1:10 with reference water).

The measurement shall be compared to reference water measurements

Positive control cycle shall be performed:
1. Every time the disposable cartridge is replaced—the first step of this cycle shall be a reference check to determine 100%
2. Upon user's request
3. Parameter—see NFREQ18_02
   REQ9_04_02: Reference water cycle

The software shall perform test cycle with reference water. The luminescence measurements at every time point shall be stored and regarded as 100% for comparison to measurements from tested source water. Reference water cycle shall be performed:
1. In first 24 hours of system's activity shall be performed at an intense frequency—parameter value defined by technician
2. Parameter defined by user see NFREQ18_02
3. Upon user's selection
4. Before testing positive control
5. After running disinfection cycle
   REQ9_04_03: Tested source water cycle

At each point the obtained level of luminescence shall be compared to the parallel time point recorded with the most recent reference water test.

Should a light level descent or ascend more than the relative limit defined percentage detected the following actions shall be performed:
1. System washing
2. Reference sampling
3. Repeat test

Should the result repeat itself—a contamination alarm shall be turned on and water sample shall be taken into “grab sample container” (if there is a place for it, the container shall be able to contain up to 2 samples).

REQ9_04_04: Drain bacteria solution chamber

The software shall drain bacteria solution chamber:
1. If new batch of hydrated bacteria shall be started
2. Upon authorized technician's request
   REQ9_05: The software shall enable system parameters change (see NFREQ18_02)
   REQ9_05_01: The software shall enable every parameter defined in system to be modified separately, from web browser and LCD screen
   REQ9_05_02: The software shall enable default parameters file insertion (by technician authority only, through web browser only). Upon “Return to default values” command (by technician authority only) the software shall set default values from file (if exists) or return to factory default values
   Feature F010—System wash

### Description

REQ10_01: The software shall perform assay chambers washing:
1. With disinfection solution once predefined timeout (parameter, see NFREQ18_02). After disinfection solution wash, the system shall be washed with water to remove disinfection substance
2. With water taken from wash container after every test cycle.
3. Number of wash cycles required for proper washing shall be a parameter (handled by authorized technician, see NFREQ18_02)

### 1.3. External Interface Requirements

External interface NF001—LAN interface

### Description

NFREQ1_01: The software shall communicate with external host PC through Ethernet

### NFREQ1_02: Configurations:

NFREQ1_02_01: DHCP: Default—DHCP disable.

The system shall enable the user (technician or admin authority!) to enable or disable DHCP. This shall be performed though LCD and web browser.

NFREQ1_02_02: IP address: Default—192.168.0.1
If DHCP disabled—the system shall enable IP address changing (by technician or admin authority user) through LCD and web browser.
NFREQ1_02_03: MAC address: Default—01234 . . . .

The system shall enable MAC address configuration (by technician user authority only) through LCD and web browser.

Probably will be performed once in device's life time

External interface NF002—GSM interface

### Description

NFREQ2_01: The software shall communicate with external cellular devices through GSM—via serial communication

### 1.4. Internal Interface Requirements

### 1.5. Usability Requirements

1. The software shall support SCADA (NIST SP800-82) in the following manner:
1. Digital Alarm Indication (On/Off)
2. Digital Malfunction indication (On/Off)
2. The software shall support stand alone and non-stand alone modes
3. The software shall support multiple—languages, according to screen restrictions. Options: European LCD (English, Spanish, French, German, Italian languages supported) and Chinese LCD (English and Chinese languages supported). The change of the language through web browser shall not apply to the language on the device and vice versa.

### 1.6. Reliability Requirements

NFREQ6_01: The software shall generate no more than one false alarm per week of continuous operation
NFREQ6_02: The tolerance of contamination determination shall be in range of +/−2%

### 1.7. Testability and Test Tools Requirements

### 1.8. Safety Requirements

### NFREQ8_01: Safety: TBD

NFREQ8_02: Security of valid operation: The software shall read and verify electronic code embedded in disposable units.
1.9. Security and privacy Requirements
NFREQ9_01: The software shall have three levels of authorization: user, technician and administrator. For exact authority definitions refer to related documents 9
NFREQ9_02: The software shall use security features for: system access, data encryption, secure communications (NIST SP800-53)

### 1.10. Performance Requirements

### 1.11. Scalability Requirements

### 1.12. Maintenance and Upgrades Requirements

NFREQ12_01: The software upgrade shall be performed using boot loader
NFREQ12_02: Future expansion in GUI interface possibilities shall be taken into consideration in SW design

### 1.13. Environmental Requirements

### 1.14. Hardware Requirements

NFREQ14_01: SPI—FPGA and external memory interface:
1. PLL shall be enabled.
2. CLKOUT shall be disabled.
3. PLL shall operate in normal mode.
4. Multiplication factor of PLL shall be MFD=0. Reduced frequency divider shall be RFD=1.
5. Master mode shall be enabled.
6. Transfer size shall be 8 bit.
7. Clock polarity shall be high.
8. Clock phase, data shall be changed on the leading edge of QSPI_CLK and captured on the following edge QSPI_CLK.
9. SPI Band Rate shall be 3.125[MHz]
10. Delay between chip select and SPI clock shall be 2.54[μs].
11. Delay after transfer shall be 5.12[μs].
12. Chip selects shall be active low.
13. End of queue pointer shall be (1111)2
14. Start of queue pointer shall be (0000)2.
15. Chip select shall return to inactive level defined by QWR[CSIV] only after the transfer of the queue entries.
16. Chip selects shall be as follows:

### NFREQ14_02: UART—GSM

| Chip select | QSPI_CS |
|---|---|
| FPGA | 0001 |
| Serial Flash | 0010 |
| Optional | 0100 |

1. Parity mode shall be: no parity.
2. Transfer size shall be 8 bits, not include start and stop bits.
3. Stop-bit length control shall be 1 bit
4. Baud rate shall be equal 38400

### NFREQ14_03: I2C—RTC and EEPROM

1. I2C clock rate shall be: Divider=576 and I2FDR=0×14.
2. I2C shall be interrupt enabled.
3. I2C module shall work as master.
4. The RTC slave address shall be “1101000”, EEPROM slave address shall be “1010010”
   NFREQ14_04: External memory
1. The memory data format shall be 8 or 16 bits
2. Access time shall be 70 ns

### NFREQ14_05: LAN

1. 100/10 Mbit/s

### NFREQ14_06: Buzzer

1. Buzzer frequency shall be 2[Khz]
2. Sound level SPL shall be 85 db

### NFREQ14_07: RTC

1. The RTC communication shall be I2C
2. The RTC Data Format shall be HH-MM-SS (hour-minute-second), month and year (00-99)
   NFREQ14_08: Disposable unit EEPROM
1. Interface with microcontroller shall be I2C
2. Data Format shall be 8 bit

### 1.15. Software Requirements

### 1.16. Development Tools Requirements

1. Utasker web server: http://www.utasker.com/index.html
2. Visual studio 2005 for web pages
3. CodeWarrior for Firmware
   1.17. Design and implementation constrains
   1.18. System requirements
   NFREQ18_01: English keyboard required while working with the system through web browser, since all the inputs to the system shall be in English.

The following inputs defined in system:
1. Password—English characters and digits only
2. E-mail address—English characters and digits
3. SMS messages file—English characters and digits
4. Parameters' values—digits
   NFREQ18_02: System parameters' limits and defaults definition:

| | Minimum | Maximum | | Default value |
|---|---|---|---|---|
| Parameter name | value | value | Resolution | for start |
| Reaction time | 5 minutes | 60 minutes | 1 minute | 15 min |
| (sample/reference/positive | | | | |
| control) | | | | |
| Temperature in bacteria chamber | 2° C. | 4° C. | 1° C. | 2° C. |
| Temperature in assay chambers | 30° C. | 32° C. | 1° C. | 31° C. |
| Disinfection frequency | every 24 hours | Every 1 hour | 1 hour | every 24 hours |
| Reference reading frequency | every 24 hours | Every 1 hour | 1 hour | Every 12 hours |
| Positive control frequency | every 7 days | Every 1 hour | 1 hour | every 24 hours |
| | (168 hours) | | | |
| Toxicity test frequency | every 24 | every 30 | 15 minute | every 15 |
| | hours (1440 | minutes | | minutes |
| | minutes) | | | |
| Background measurement | 1 | 96 | 1 per number | 24 |
| frequency | | | of toxicity | |
| | | | tests | |
| Luminescence ascend/descend % | 1 | 100 | 1% | 50% |
| Number of wash cycles | 1 | 5 | cycle | 2 |
| Temperature failure time | 1 hour | 24 hours | 1 hour | 1 hour |
| Initial light read | TBD | TBD | Photon/counts | TBD |

NFREQ18_03: Parameters change and command execution authorities:

| | End user | | Local administrator | | Service technician | |
|---|---|---|---|---|---|---|
| Parameter | view | change | view | change | view | change |
| Temperature | Y | N | Y | N | Y | Y |
| (bacteria | | | | | | |
| & assay | | | | | | |
| chambers) | | | | | | |
| Reaction time | Y | N | Y | Y | Y | Y |
| Frequency of | Y | N | Y | Y | Y | Y |
| tests (organic/ | | | | | | |
| metal) | | | | | | |
| Positive | Y | Y | Y | Y | Y | Y |
| control cycle | | | | | | |
| Disinfection | Y | N | Y | Y | Y | Y |
| cycle | | | | | | |
| Wash cycle | Y | N | Y | N | Y | Y |
| Reference | Y | N | Y | Y | Y | Y |
| cycle | | | | | | |
| Initiate new | Y | Y | Y | Y | Y | Y |
| hydration of | | | | | | |
| bacteria vial | | | | | | |
| Drain bacteria | — | N | — | N | — | Y |
| solution | | | | | | |
| chamber | | | | | | |

1-97. (canceled)

98. A disposable cartridge 100 for use in assaying an analyte in an automated water quality monitoring analyzer 1000 configured for conveying at least one reaction chamber 30 to predetermined positions for enabling fluid transfer of analytes, bacteria and reagents into said reaction chamber 30 and provision of emitted light to said automated water quality monitoring analyze 1000; said disposable cartridge comprising;
a. a platform 50;
b. an array of containers 60 for separately containing at least one reagent, and at least one buffer; said containers being mounted on said platform;
c. a fluid transferring means 70 for fluid transfer and mixing between said array of containers, said at least one reaction chamber, and said analyzer;
said;
characterized in that said array comprises at least one container containing a freeze dried preparation of luminescent bacteria; said at least one buffer and said at least one hydrated preparation of freeze-dried luminescent bacteria are conveyable to said reaction chamber in a predetermined sequence such that luminescent light detectable by said analyzer is emitted.

99. The cartridge according to claim 98 wherein at least one of the following holds true; said array additionally comprises at least one container containing disinfection buffer (DBC) mounted on said platform, said array additionally comprises at least one container containing reference water (RWC) mounted on said platform, and said array additionally comprises a chamber for bacterial suspension.

100. The cartridge according to claim 98 wherein at least one of the following holds true; any of said containers are provided with volume sensors, said sensors provided with logical coupling means to a microprocessor in said analyzer, said sensors are adapted to signal data concerning contents of said containers and said reaction chamber to said microprocessor in said analyzer.

101. The cartridge according to claim 98 wherein said platform is movable via means selected from the group consisting of a gantry, rail, lift, carriage, or track.

102. The cartridge according to claim 98 wherein at least one of the following holds true; said containers, chambers and fluid transferring means is adapted for manipulation by mechanical means, said array of containers and said at least one reaction chamber are attached by locking and mounting means to said platform at specific locations on said platform, array of containers additionally comprises at least one container for containing storage buffer, said array of containers additionally comprises at least one container for containing organic buffer, said organic buffer optimized for use in assays of organic analytes, said array of containers additionally comprises at least one container for containing a metallic buffer, said metallic buffer optimized for use in assays of cationic heavy metals and metalloid analytes, said array of containers are provided with reagents and buffers adapted for use in assays selected from a group consisting of fluorometric assays, chemiluminescent assays and colorimetric assays, said platform is adapted to lock into said analyzer in a predetermined configuration, said platform is adapted to be temperature controlled at specific container and reaction chamber mounting locations, thereby holding said containers and said chamber at predetermined temperatures, said platform is adapted to be cooled at specific locations, so as to facilitate cooling of said containers and said at least one reaction chamber to predetermined temperatures.

103. The cartridge according to claim 98 wherein said fluid transferring means are selected from a group consisting of inlet ports, outlet ports, syringes, valves, taps and connectors.

104. The cartridge according to claim 98 wherein said cartridge is configured for storage in a module of said analyzer for predetermined periods of about one month.

105. The cartridge according to claim 98 wherein said cartridge is configured for storage in said module of said analyzer for a period of about 1-3 months.

106. The cartridge according to claim 98 wherein said cartridge is provided in kit form for assembly by the user according to instructions associated with said kit.

107. A method of assaying an analyte in an automated water quality analyzer wherein said method comprises steps of;
a. obtaining a disposable cartridge comprising;
i. a platform 50;
ii. an array of containers 60 for separately containing at least one reagent, at least one buffer and at least one freeze dried preparation of luminescent bacteria, said containers being mounted on said platform;
iii. ,
iv. fluid transferring means 70 for fluid transfer and mixing between said array of containers, said at least one reaction chamber, and said analyzer;
said reaction chamber 30 is conveyable to predetermined positions for enabling fluid transfer of said analyte, bacteria and reagents into said reaction chamber 30 and provision of said emitted light to said automated water quality monitoring analyzer 1000.
b. installing said cartridge into said automated water quality monitoring analyzer;
c. operating said automatic analyzer thereby conveying said reaction chamber (30) to said predetermined locations in said analyzer while simultaneously sequentially manipulating contents of said containers and conveying said contents to said reaction chamber so as to activate said luminescent bacteria such that light is emitted;
d. detecting said emitted light;
e. processing the results obtained thereby assaying said analyte.
wherein said light to be detectable is luminescence provided by said at least one buffer and said at least one freeze dried preparation of luminescent bacteria conveyed to said reaction chamber in a predetermined sequence.

108. A method of assaying an analyte in an automated water quality analyzer according to claim 107 wherein at least one of the following holds true, said array additionally comprises at least one container containing disinfection buffer (DBC) mounted on said platform, said array additionally comprises at least one container containing reference water (RWC) mounted on said platform, said array additionally comprises a container for containing a bacterial suspension.

109. A method of assaying an analyte in an automated water quality analyzer according to claim 107 wherein at least one of the following holds true; said containers are provided with volume sensors, said sensors provided with logical coupling means to a microprocessor in said analyzer, said sensors are adapted to signal data concerning contents of said containers and said at least one reaction chamber to said microprocessor in said analyzer, said platform is adapted for traveling on a gantry, rail, lift, carriage or track.

110. A method of assaying an analyte in an automated water quality analyzer according to claim 107 wherein at least one of the following holds true; said containers, at least one chamber and said fluid transferring means are adapted for manipulation by mechanical means, said containers, said at least one chamber, and said fluid transferring means are attached by locking and mounting means to said platform at specific locations on said platform.

111. A method of assaying an analyte in an automated water quality analyzer according to claim 107 wherein at least one of the following holds true; said platform is adapted to lock into said analyzer in a predetermined configuration, said platform is adapted to be temperature controlled at specific container and chamber mounting locations, thereby holding said containers and said at least one chamber at predetermined temperatures, said platform is adapted to be cooled at specific container locations, thereby cooling said containers and said at least one chamber to predetermined temperatures, said fluid transferring means are selected from a group consisting of inlet ports, outlet ports, syringes, valves, taps and connectors.

112. A method of assaying an analyte in an automated water quality analyzer according to claim 107 wherein said array of containers additionally comprises at least one container filled with storage buffer.

113. A method of assaying an analyte in an automated water quality analyzer according to claim 107 wherein at least one of the following holds true; said array of containers additionally comprises at least one container for organic buffer, said organic buffer optimised for use in assays of organic analytes, said array of containers additionally comprises at least one container for metallic buffer, said metallic buffer optimized for use in assays of cationic heavy metals and metalloids analytes.

114. A method of assaying an analyte in an automated water quality analyzer according to claim 107 wherein said cartridge is adapted for storage in a module of said analyzer for predetermined periods of about one month.

115. A method of assaying an analyte in an automated water quality analyzer according to claim 107 wherein said cartridge is adapted for storage in said module of said analyzer for a period of about 1-3 months.

116. The method according to claim 107 wherein said method comprises further steps of obtaining said cartridge and operating said analyzer so as to activate automated steps of;
a. transferring aliquots of storage buffer into a predetermined number of said containers containing said freeze dried preparations of luminescent bacteria;
b. incubating said freeze dried preparations of luminescent bacteria with said aliquots of said storage buffer so as to ensure hydration and biological activation of said bacteria;
c. mixing said freeze dried preparations of luminescent bacteria with said aliquots of said storage buffer;
d. adding organic or metal buffer to said reaction chamber;
e. adding test water to said reaction chamber;
f. mixing said water for about 2 seconds to homogenize said water and said buffers;
g. adding said bacteria to said reaction chamber;
h. detecting said emitted light;
i. processing said results obtained, thereby assaying said analyte.

117. A method of assaying an analyte in an automated water quality analyzer according to claim 107 wherein said array of containers are provided with reagents and buffers adapted for use in assays selected from a group consisting of fluorometric assays, chemiluminescent assays and colourimetric assays.
